(19) United States
(12) Patent Application Publication
(10) Pub. No.: US 2006/0173219 A1
(43) Pub. Date: Aug. 3, 2006

(54) Protective groups for crossed aldol condensations

(76) Inventor: Christian Everett, Springfield, IL (US)

Correspondence Address: CHRISTIAN EVERETT, 483 SOUTH KIRKWOOD ROAD, # 228, SAINT LOUIS, MO, 63122, US

(21) Appl. No.: 11/049,144

(22) Filed: Feb. 3, 2005

(51) Int. Cl. C07C 45/45; C07F 1/02

(52) U.S. Cl. 568/345

(57) ABSTRACT

A process for protecting aldehydes and ketones in crossed aldol condensations where both aldols possess alpha-carbon hydrogens, comprising protecting the target aldol by forming an acetal or imine with an alcohol, glycol or primary amine which may contain electron-withdrawing groups, adding a base of sufficient strength to abstract a proton from the alpha-carbon of the acetal or imine and adding the second aldol to form the salt of the saturated hydroxy addition compound, thereby minimizing by-products, waste and separation difficulties, then forming the unsaturated addition compound and decomposing the acetal or imine with dilute acid. This process having the advantage that a ketone may be added to a protected aldehyde target to produce an aldehyde as a product which was not previously possible.

### BACKGROUND

1. Field of Invention

This invention relates to aldol condensations, especially to condensations of two different aldols.

2. Description of Prior Art

In order to produce new compounds two aldol compounds are often reacted in the known manner. If one of these compounds does not have an alpha-carbon with hydrogen on it (benzaldehyde, formaldehyde, tert-pentaldehyde) the reaction produces one product. However, if both aldols have hydrogen on the alpha-carbon specifity is lost and all four possible products result.

For example, equiniolar amounts of acetaldehyde and propionaldehyde react to produce equimolar quantities of 2-butenal, 2-pentenal, 2-butene-2-methyl-al, and 2-pentene-2methyl-al.

Attempts have been made to enhance specifity by increasing the amount of one of the reactants to 30:1 (U.S. Pat. No. 6,028,231) but this still produces byproducts and waste and is impractical where both aldols are approximately equal in cost.

Also there is currently no method for a ketone to add to an aldehyde to produce an aldehyde product. When the compounds are mixed the more reactive aldehyde always adds to the ketone producing a ketone as a product.

### OBJECTS AND ADVANTAGES

Several objects and advantages of the present invention are:
(A) Increased yield of product
(B) Less waste-no side reactions
(C) Ease of purification of product
(D) Stable under basic conditions of condensation
(E) Decomposed under mildly acid conditions after condensation
(F) Alcohols and glycols used to form acetals and amines used to form imines may be recycled, reducing overall costs
(G) Allows formation of ketone-aldehyde compounds previously available only by many low-yield steps

### SUMMARY

In accordance with the present invention a protective group consisting of an acetal or an imine, which may contain electron-withdrawing functions, for crossed aldol condensations: whereby the carbonyl oxygen of the protected aldol is rendered unreactive to allow the carbonyl oxygen of the unprotected aldol to react with the alpha-carbon hydrogens of the protected aldol, in the presence of a base of sufficient strength, to produce one product compound with no side reactions.

### DESCRIPTION

The ability of a compound (nitroparafins, alkyl cyanides, other aldehydes and ketones) to react with aldehydes and ketones is a function of the acidity of the hydrogen on the carbon alpha to the functional group. This acidity is described by the term pKa. In the case of acetone (pKa 20 in water), as typical of aldehydes and ketones, the ability to delocalize the positive charge of an alkali metal base is largely due to the conjugation of the carbonyl bond. Resonance between the ketonic and enolic structures of acetone allow the molecule to absorb the positive charge of the alkali metal ion.

When an acetal is formed to render the carbonyl oxygen unreactive so other portions of the molecule may be modified, the double bond is replaced by two single bonds, conjugation is lost and acidity of the alpha hydrogens decreases markedly (pKa increases).

Alkali metal hydroxides in water serve only to condense compounds with a pKa up to about 30. Above this value stronger bases must be used to initiate the reaction by abstracting a proton from the target compound. Sodium ethoxide in ethanol will serve to condense compounds with a pKa up to about 33, sodium di-tert-butylamine in di-tert-butylamine will initiate condensation with compounds with a pKa up to about 38, and butyl-lithium in hexane will abstract a proton from a hydrogen with a pKa up to about 45.

Even without conjugation, the two ether linkages on the functional carbon of an acetal are fairly powerful electron-withdrawing groups. This electron-withdrawing function can be increased, increasing the acidity of the alpha-hydrogens, by using alcohols and glycols that possess electron-withdrawing groups to form the acetal, these being typified by perfluoro-tert-butanol and perfluoro-pinacol.

By using these two techniques of employing stronger bases and electron-withdrawing alcohols and glycols, either singly or in tandem, an acetal can be made to condense with an aldehyde or ketone in the same manner as an unprotected aldehyde or ketone without side reactions occurring.

Alternately, conjugation may be preserved by forming an imine from the target aldehyde or ketone with a primary amine. Although the imine possesses a double bond, nitrogen is a more electropositive element than oxygen. This may be offset by using a primary amine possessing electron-withdrawing groups, typified by tri-fluoro methylamine and per-fluoro tert-butylamine. The imine group is less preferred to the acetal group because under certain conditions it may polymerize over time. Once the imine is formed it may be reacted with another aldehyde or ketone in the presence of a base of sufficient strength in the same manner as an unprotected aldehyde or ketone without side reactions occurring.

### EXAMPLE 1

To 0.1 moles of acetaldehyde-perfluoropinacol-acetal (32.9 grams) in 250 ccs of diethylamine At 0 C is added 0.1 moles of sodium diethylamine (9.5 grams) in 100 ccs of diethylamine at 0 C with stirring. Five minutes are allowed for the sodium anion of the acetaldehyde-perfluoropinacol-acetal to form, whereupon 0.1 moles of propionaldehyde (6 grams) at 0 C is added over 3 minutes with stirring to form the sodium salt of 3-hydroxy pentanal-perfluoropincol-acetal. The diethylamine is removed under reduced pressure and the solid salt is gradually added to dilute hydrochloric acid at 0 C to form 2-pentene-al-perfluoropinacol-acetal. The reaction mixture may now be heated to 100 C to decompose the acetal yielding pure 2-pentene-al

### EXAMPLE 2

To 0.1 moles of 1,3 propion-dialdehyde-perfluoro-tert-butyl alcohol-acetal (99.2 grams) in 500 ccs of ethanol at 0 C is added 0.1 moles of sodium ethoxide (6.8 grams) in 50 ccs of ethanol at 0 C with stirring. Five minutes are allowed for the sodium anion of 1,3 propion-dialdehyde-perfluoro-tert-butyl alcohol acetal to form whereupon 0.1 moles of cyclohexanone at 0 C is added over 3 minutes with stirring to form the sodium salt of 1-hydroxy cyclohexane 1-(2) 1,3 propion-dialdehyde-perfluoro-tert-butyl alcohol-acetal. The ethanol is removed under reduced pressure and the solid salt is gradually added to dilute hydrochloric acid

at 0 C to form delta-2 cyclohexane, 1,3 propion-dialdehyde-perfluoro-tert-butyl alcohol-acetal. The reaction mixture may now be heated to 100 C to decompose the acetal yielding pure delta-2 cyclohexane 1,3 propion-dialdehyde.

### EXAMPLE 3

To 0.1 moles of acetaldehyde-perfluoro-tert-butylamine-imine (24.9 grams) in 250 ccs of di-tert-butylamine at 0 C is added 0.1 moles of sodium di-tert-butylamine (15.6 grams) in 100 ccs of di-tert-butylamine at 0 C with stirring. Five minutes are allowed for the sodium anion of acetaldehyde-perfluoro-tert-butylamine-imine to form whereupon 0.1 moles of acetone (7 grams) at 0 C are added over 3 minutes with stirring to form the sodium salt of 3-hydroxy, 3-methyl butanal-perfluoro-tertbutylamine-imine. The di-tert-butylamine is removed under reduced pressure and the solid salt is gradually added to dilute hydrochloric acid at 0 C to form 3-methyl butene-al-perfluoro-tert-butylamine-imine. The reaction mixture may now be heated to 100 C to decompose the imine to yield pure 3-methyl butene-al.

### EXAMPLE 4

To 0.1 moles of acetaldehyde-ethylene glycol-acetal (8.7 grains) in 250 ccs of hexane at −20 C is added 0.1 moles of butyl-lithium (6.4 grams) in 100 ccs of hexane at −20 C with stirring. Five minutes are allowed for the lithium anion of acetaldehyde-ethylene glycol-acetal to form whereupon 0.1 mole of 1,5 dimethyl, 5-hexene-al (10.9 grams) in 50 ccs of hexane at −20 C is added over 3 minutes with stirring to form the lithium salt of 3,7 dimethyl, 3-hydroxy, 7 octene-al-ethylene glycol-acetal. The hexane is removed under reduced pressure and the solid salt is added gradually to dilute hydrochloric acid at 0 C to form citral-ethylene glycol-acetal. The reaction mixture may now be heated to 100 C to decompose the acetal to yield pure citral.

### CONCLUSION, RAMIFICATION AND SCOPE

Accordingly, the reader will see the acetal and imine protective groups for crossed aldol condensations are more convenient than current methods. Furthermore the process has additional advantages in that:

Yields are increased

By-products and waste are reduced

Separation from by-products is easier

Allows deactivation of aldehyde carbonyl oxygen so a ketone may add to the aldehyde's alpha-carbon hydrogen's to produce an aldehyde as a product.

Although the description contains specifities, these should not be construed as limiting the scope of the invention, but merely providing illustrations of some of the presently preferred embodiments of the invention. For example other alcohols, glycols and amines may be used, and other bases, solvents and acids may be used. Thus the scope of the invention should be determined by the appended claims and their legal equivalents rather than by the examples given.

I claim:

1: A method for improving the yield of crossed aldol condensations comprising the steps of
A: Forming an acetal of the aldol losing its alpha-carbon hydrogens of the reaction.
B: Adding a base with a pKa greater than the pKa of the alpha-carbon hydrogens of the acetal, such as alkali metal alkoxides, alkali metal alkoxides, alkali metal dialkyl-amines, or butyl-lithium.
C: Adding the aldol losing its carbonyl oxygen in the reaction to form the salt of the saturated hydroxy addition compound of the acetal
D: Forming the unsaturated addition compound and decomposing the acetal with dilute acid.

2: A method for improving the yield of crossed aldol condensations comprising the steps of
A: Forming an amine of the aldol losing its alpha-carbon hydrogens in the reaction.
B: Adding a base with a pKa greater than the pKa of the alpha carbon hydrogens of the imine, such as alkali metal alkoxides, alkali metal dialkyl-amines, or butyl-lithium.
C. Adding the aldol losing its carbonyl oxygen in the reaction to form the salt of the saturated hydroxy addition compound of the imine.
D: Forming the unsaturated addition compound and decomposing the imine with dilute acid.

3: A method for improving the yield of crossed aldol condensations comprising the steps of
A: Forming an acetal or imine of the aldol losing its alpha carbon hydrogens using an alcohol or glycol possessing electron withdrawing groups, such as perfluoro-tert-butanol or perfluoro-pinacol or a primary amine possessing electron withdrawing groups, such as trifluormethyl-amine or perfluoro-tert-butyl-amine.
B: Adding a base with a pKa greater than the pKa of the alpha carbon hydrogens of the acetal or imine, such as alkali metal alkoxides, alkali metal dialkyl-amines or butyl-lithium.
C: Adding the aldol losing its carbonyl oxygen in the reaction to form the salt of the saturated hydroxy addition compound of the acetal or imine.
D: Forming the unsaturated addition compound and decomposing the acetal or imine with dilute acid.
